(19) United States
(12) Patent Application Publication
(10) Pub. No.: US 2010/0278738 A1
(43) Pub. Date: Nov. 4, 2010

(54) METHOD TO DETECT AND MONITOR ISCHEMIA IN TRANSPLANTED ORGANS AND TISSUES

(76) Inventors: Thomas J. Sitzman, Middleton, WI (US); Karol A. Gutowski, Glenview, IL (US)

Correspondence Address: Intellectual Property Dept./Dewitt Ross & Stevens;Wisconsin Alumni Research Foundation, 2 East Mifflin Street,, Suite #600, Madison, WI, 53703-2865, US

(21) Appl. No.: 12/773,389

(22) Filed: May 4, 2010

Related U.S. Application Data
(60) Provisional application No. 61/175,314, filed on May 4, 2009.

(51) Int. Cl. A61K 49/00; A61P 9/10

(52) U.S. Cl. 424/9.1

(57) ABSTRACT

Disclosed is a method of detecting and/or monitoring ischemia in a tissue or organ, such as a free flap transfer. The method includes the steps of measuring in real time or near-real time interstitial glucose concentration, or rate of change of interstitial glucose concentration over time, or both, in the tissue or organ. A reduced glucose concentration or a negative rate of change of glucose concentration in the tissue or organ as compared to a control glucose concentration or rate of change indicates ischemia in the tissue or organ.

### CROSS-REFERENCE TO RELATED APPLICATIONS

Priority is hereby claimed to provisional application Ser. No. 61/175,314, filed May 4, 2009, which is incorporated herein by reference.

### BACKGROUND

A free tissue transfer (or a “free flap” transfer) is a procedure wherein an isolated and specific region of the body (for example, skin, fat, muscle, or bone), and its associated vasculature, is excised from one region of the body and transferred to another region of the body. The excised tissue is then reattached and the arterial and venous vessels reattached to establish circulation in the transferred tissue. This ability to transplant living tissue from one region of the body to another has greatly facilitated the reconstruction of complex defects. As used herein, the terms “free flap” and “free tissue transfer” are synonymous. Both terms are used to describe the movement of tissue from one site on the body to another. The word “free” indicates that the tissue, along with its blood supply, is detached from the original location (the donor site) and then transferred to another location (the recipient site).

Free tissue transfer has become commonplace in many centers around the world. The numerous advantages include stable wound coverage, improved aesthetic and functional outcomes, and minimal donor site morbidity. Since the introduction of free tissue transfer in the 1960s, the success rate has improved substantially

Since the time free tissue transfers were first developed, it has become possible to surgically repair increasingly larger and more complex tissue defects. For example, breast reconstruction after a mastectomy is now an essentially routine procedure. However, despite myriad advances in surgical techniques and instrumentation, ischemia and the subsequent necrosis of the transferred tissue remains problematic in a small, but significant percentage of patients. The same is true when transplanting other tissues and organs. It has been more than 50 years since the first successful kidney transplant (in 1954), yet ischemia in transplanted tissue continues to be a root cause of morbidity and mortality in a significant number of patients.

As used herein, the term “ischemia” means a restriction in blood supply due to any cause. In the context of a free tissue transfer, ischemia generally takes the form of clotting in the blood vessels after the flap is excised from the donor site and prior to, or just after, the flap is transplanted at the recipient site. Ischemia damages the tissue, with the ultimate result being tissue necrosis. Ischemia thus requires that the transplanted tissue be removed.

Conventionally, in the free tissue transfer of skin, the patient is monitored post-surgery to detect ischemia in the transplanted tissue. This typically involves a gross inspection of the transplant for color, temperature, and pulse, as well as stethescopic or ultrasonic auscultation to detect arterial and venous blood flow. The primary drawback of simply monitoring the patient in this fashion is that by the time any ischemia is detected (typically within 72 hours after surgery), it is often too late to do anything about it. At that point, the only course of action is to remove the transplanted tissue.

There are, however, two instrumental approaches that have been used to monitor tissue ischemia. The first approach uses blood oximetry; the second approach uses micro-dialysis. Both approaches, however, suffer from drawbacks in terms of cost, sensitivity, and real-time functionality.

ViOptix, Inc., of Fremont, Calif., markets a proprietary tissue oximetry technology which enables non-invasive, direct, real-time measurement of local tissue oxygen saturation. Oxygen, of course, is a key parameter in many clinical areas such as tissue viability, revascularization, cancer management, circulatory exploration and muscle assessment. The level of oxygen saturation can thus be used to measure tissue ischemia. See, for example, U.S. Pat. No. 7,247,142, issued Jul. 24, 2007, and U.S. Pat. No. 7,525,647, issued Dec. 21, 2007, both of which are assigned to ViOptix, Inc.

Micro-dialysis is a technique used to determine the chemical components of the fluid in the extracellular space of tissues. A microdialysis probe, which is inserted into the tissue, is a tiny tube made of a semi-permeable membrane. A dialysate solution is pumped through the dialysis probe, and chemical entities in the extracellular space diffuse into the dialysate. The dialysate is then collected and analyzed to determine the identities and concentrations of molecules that were in the extracellular fluid. In the context of free tissue transfers, it has been found that simultaneously monitoring the level of glucose, lactate, pyruvate and glycerol correlates well with the presence of ischemia in the free tissue. See, for example, Röjdmark et al. (January 1992) “Comparing metabolism during ischemia and reperfusion in free flaps of different tissue composition,” European Journal of Plastic Surgery 24(7): 349-355. In this study, the interstitial kinetics of glucose, lactate, pyruvate and glycerol were studied during ischemia and reperfusion in human free flaps of different tissue compositions (skin, muscle and adipose tissue). The concentrations of the substances were determined repeatedly during ischemia and reperfusion, and laser doppler flowmetry was used to document revascularization. Microdialysis catheters were placed in the flap tissue and in similar, non-operated control tissue. The drawback of micro-dialysis, however is that it is not a real-time protocol. Like conventional visual monitoring, it can detect ischemia, but not soon enough to do anything about it.

Thus there remains a long-felt and unmet need for a sensitive, accurate, and real-time method to detect and to monitor ischemia in a transplanted tissue or organ.

### BRIEF DESCRIPTION OF THE DRAWINGS

FIG. 1 is a graph depicting interstitial glucose levels (mg/dL) vs. time (in minutes) for all vessel occlusions for all animals tested. Legend: (-♦-) dorsal control; (-▪-) occluded flap; (-▴-) control flap. Animals were euthanized after 90 minutes and glucose monitoring continued until 120 minutes.

FIG. 2 is a graph depicting interstitial glucose levels (mg/dL) vs. time (in minutes) after arterial occlusion. Legend: (♦) dorsal control; (-▪-) occluded flap; (-▴-) control flap. Animals were euthanized after 90 minutes and glucose monitoring continued until 120 minutes.

FIG. 3 is a graph depicting interstitial glucose levels (mg/dL) vs. time (in minutes) after venous occlusion. Legend: (-♦-) dorsal control; (-▪-) occluded flap; (-▴-) control flap. Animals were euthanized after 90 minutes and glucose monitoring continued until 120 minutes.

FIG. 4 is a graph depicting the combined results depicted in FIGS. 3 and 4. Legend: (-♦-) dorsal control; (-▪-) arterial occluded flap; (--) venous occluded flap; (-▴-) control flap.

### DETAILED DESCRIPTION OF THE INVENTION

Ischemia occurs when the blood supply to tissues is blocked and can be caused by a variety of trauma or disease. The lack of blood supply prevents oxygen from getting to cells and prevents waste products from being removed out of the tissue. In plastic surgery, blood vessel occlusion after free tissue transfer leads directly to transfer ischemia. This ischemia must be corrected within six hours of vessel occlusion to prevent complete tissue flap loss. If detected early enough, vessels can be unblocked, and the tissue transfer can be saved. However, for the ischemia to be addressed, it is imperative to detect where and when the blockage is occurring as quickly as possible in order to target treatment. The same situation applies in other transplant procedures. For example, early detection of thrombosis after kidney transplant can salvage the transplanted organ if blood flow is re-established quickly. Again, the ischemia must be detected quickly to have any probability of salvaging the transplant. Similarly, monitoring ischemia in the brain after stroke or other neurological trauma can affect the strategy selected regarding re-perfusion of the damaged area. (Repeat cycles of ischemia and re-perfusion can cause further damage to the brain of a stroke patients and should be avoided.) Re-perfusion needs to be tightly controlled in these patients. That can only be done if ischemia can be monitored quickly, precisely, and accurately, in real time or near real time.

The present invention is a method of monitoring absolute glucose levels and the rate of change in glucose levels in tissue. It has been found that both the absolute glucose level and the rate of change of glucose level in tissue correlates very closely with the onset and progression of ischemia in the tissue. Using a composite log regression of both variables enables ischemia to be detected with a speed, accuracy, and precision previously unattainable. The method can be implemented using absolute glucose level as the metric, or rate of change of glucose level as the metric, or both glucose level and rate of change as the metric (preferred).

Logistical regression (i.e., log regression) analysis is well-known and will not be described in great detail herein. Very briefly, a log regression analysis uses the following logistic function: ƒ(z)=1/1+e^{-z}. The logistic function can take as a numerical input any value from negative infinity to positive infinity. Because of the logarithmic nature of the function ƒ(z), the output is confined to values between 0 and 1. The variable z represents the exposure to a pre-selected set of independent variables (in this case absolute glucose level and/or rate of change of glucose level). The function ƒ(z) represents the probability of a particular outcome (ischemia, necrosis, etc.) given the set of explanatory variables. The variable z is a measure of the total contribution (i.e., the composite value) of all the independent variables used in the model.

The variable z is usually defined as z=β₀+β₁x₁+β₂x₂+β₃x₃+ . . . β_{y}x_{y}, wherein β₀ is called the “intercept” and β₁, β₂, β₃, etc. and so on, are called the “regression coefficients” of x₁, x₂, and x₃, respectively. The intercept is the value of z when the value of all independent variables is zero (e.g., the value of z in a transplant with no risk of ischemia). Each of the regression coefficients describes the size of the contribution of that specific risk factor. A positive regression coefficient means that that explanatory variable increases the probability of the outcome, while a negative regression coefficient means that variable decreases the probability of that outcome; a large regression coefficient means that the risk factor strongly influences the probability of that outcome; while a near-zero regression coefficient means that that risk factor has little influence on the probability of that outcome. Logistic regression is a useful way of describing the relationship between one or more independent variables (e.g., absolute glucose level, rate of change of glucose level) and a binary response variable, expressed as a probability, that has only two possible values (i.e., ischemia in the transplanted tissue/organ or no ischemia). For an exhaustive treatment, see, for example, “Statistics, 4th Edition,” David Freedman, Robert Pisani, and Roger Purves, © 2007, W. W. Norton & Company, ISBN-13: 978-0393929720.

Importantly, the method described herein accurately detects and monitors both arterial and venal occlusion. Detection times are very rapid, within minutes of the onset of ischemia. This is a stark contrast to microdialysis, wherein the tissue must be monitored at specific time intervals for hours. Microdialysis also suffers from having poor specificity in determining venal occlusion. The present method measures arterial occlusion with 100% sensitivity and specificity in 20 minutes or less. The method likewise detects venous occlusion with equal success.

In the preferred version of the method, glucose level and rate of change are measured using a continuous glucose monitor, such as a “Guardian”-brand glucose monitor, which is manufactured and marketed worldwide by Medtronic, Inc., Minneapolis, Minn. The “Guardian”-brand glucose monitoring device is described in U.S. Pat. No. 6,809,653, incorporated herein by reference, and in the manufacturer's User's Guides, also incorporated herein by reference. (The manufacturer's User's Guides are included as part of corresponding provisional application Ser. No. 61/175,314, filed May 4, 2009.) Several other suitable devices are commercially available, such as DexCom's “Seven Plus”-brand glucose monitor (DexCom, Inc., San Diego, Calif.), Medtronics' “Paradigm”®-brand real-time glucose monitoring system (Medtronic Diabetes, Northridge, Calif.), and Abbott's “FreeStyle Navigator”®-brand glucose monitoring system (Abbott Diabetes Care, Inc., Alameda, Calif.). See also U.S. Pat. Nos. 5,390,671; 5,391,250; 5,568,806; 5,586,553; 5,777,060; 5,779,665; 5,786,439; 5,851,197; 5,882,494; 5,954,643; 6,093,172; 6,293,925; 6,462,162; 6,520,326; 6,607,509, 7,693,560; 7,657,297; 7,654,956; 7,651,596; 7,640,048; 7,637,868; 7,632,228; 7,615,007; 7,613,491; 7,599,726; 7,591,801; 7,462,264 7,225,535; 7,670,853; 7,381,184; 7,550,069; 7,563,350; 7,582,059; 7,003,340; 7,510,564; and 7,583,990, all of which are incorporated herein by reference. Note, however, any device capable of and dimensioned and configured for measuring interstitial glucose on a continuous or semi-continuous basis may be used in the present method, whether now known or developed in the future.

The “Guardian”-brand glucose monitor includes a sensor (part nos. MMT-7002 or MMT 7003) which is a membrane-covered electrode that measures glucose levels in the interstitial space where the sensor is inserted. The sensor is operationally connected to a transmitter (part no. MMT-7703). The transmitter sends the glucose data gathered by the sensor to a monitor (part nos. CSS-7100 or CSS7100K) that displays (and stores) real-time glucose measurements, change in glucose levels, historic glucose levels, high and low glucose levels, etc. A USB-compatible, wireless radio frequency upload device (part no. MMT-7305) can also be used to download data from the transmitter directly to a programmable computer. Using this device, glucose levels in a free-flap tissue or organ to be transplanted can be transferred in real time to a computer and monitored automatically and continuously (again in real time) and an alarm sounded (automatically) if the absolute glucose level detected by the sensor dips below a preset level, or the rate of change of the glucose level exceeds a preset level.

Thus, one version of the invention is directed to a method of detecting and/or monitoring ischemia in a tissue or organ. The method comprises measuring in real time or near-real time glucose concentration in the tissue or organ. A reduced glucose concentration in the tissue or organ as compared to a control glucose concentration indicates ischemia in the tissue or organ. As used herein, the term “control” is used in its broadest sense to mean a “normal” or “acceptable” glucose level or a range of “normal” or “acceptable” glucose levels established on a patient-by-patient, tissue-by-tissue, and/or organ-by-organ basis. The control may also or alternatively be based on aggregate glucose values taken from a sampling of “normal patients,” “normal tissues,” and/or “normal organs” and presented in the form of a standard curve or a set of standard values that constitute acceptable glucose levels and acceptable rates of change of glucose levels. The control values are then used to establish what constitutes unacceptably low glucose levels and/or unacceptably steep rates of change in glucose levels that are indicative of ischemia in the tissue or organ to be transplanted. Control glucose values can be obtained directly from the donor patient (for an autologous free-flap transfer) or donor tissue/organ prior to transplantation (by sampling the tissue or organ to be transplanted). Control glucose values may also be aggregated from the values taken from many different patients. The control values may be obtained in advance (e.g., by compiling a standard curve of glucose values) or contemporaneously with the treatment protocol being undertaken. In short, as used herein “control” means any protocol designed to provide a reference set of glucose level data which can be compared with data obtained from the tissue or organ that is being transplanted to thereby determine whether the glucose values in the tissue or organ are within an acceptable range prior to, during, and after transplantation. or whether the levels have reached a point indicating that ischemia has taken place in the tissue or organ (and thus further medical action must be taken to relieve the ischemia before cell death occurs).

Likewise, the invention includes a method of detecting and/or monitoring ischemia in a tissue or organ comprising measuring the rate of change of glucose concentration in the tissue or organ. Here, a negative rate of change of glucose concentration in the tissue or organ (as compared to a control glucose concentration) indicates ischemia in the tissue or organ. More specifically, the method disclosed herein is particularly useful to detect and/or monitor ischemia in a free tissue transfer.

### EXAMPLES

The following Examples are presented to provide a more complete description of the method disclosed and claimed herein. The Examples do not limit the scope of the claimed method in any fashion.

Bilateral vertical rectus abdominus myocutaneous (VRAM) flaps were raised on the superior epigastric vessels in adult male Sprague Dawley rats. The abdomen of each test animal was shaved and bilateral VRAM flaps were marked. Medtronic “Guardian”-brand real-time glucose monitors where then put in place on each flap. The bilateral VRAM flaps were then elevated based solely on the vascular pedicle.

Interstitial glucose monitoring was then performed using the Medtronic monitors. Glucose was measured at 5-minute intervals and the data compiled electronically.

Each animal was then further manipulated to have an occluded flap and a control flap. Animals were divided into groups to have either an arterial occlusion or a venous occlusion. In the arterial occlusion flaps, the superior epigastric artery was selectively ligated and divided. (Preliminary experiments demonstrated that division of the artery was necessary to completely eliminate arterial blood flow.)

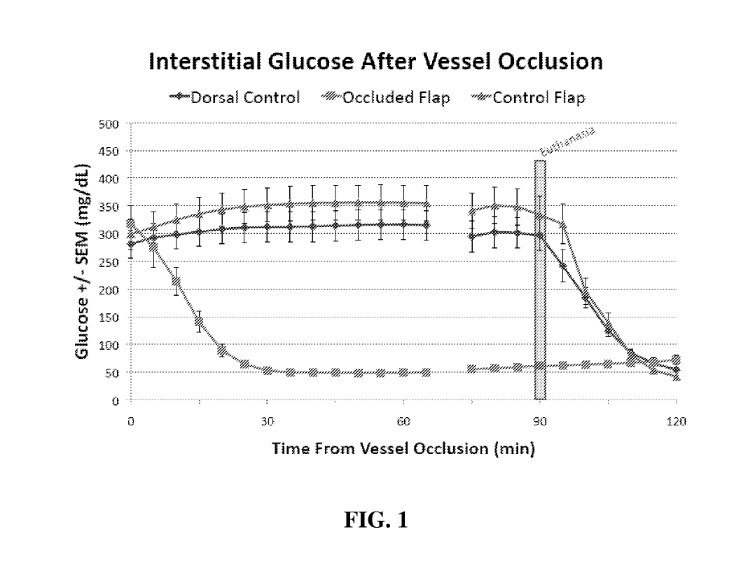

Surgery was performed on 22 rats. One (1) control flap was excluded due to pedicle injury during elevation. Four (4) venous occlusion flaps were excluded due to venous bleeding from within the flap after ligation of the superior epigastric vein. Thus, the total number of flaps analyzed was as follows: 21 control flaps (elevation only, no occlusion); 10 arterial occlusion flaps (elevation with arterial occlusion); and 8 venous occlusion flaps (elevation with venous occlusion). The interstitial glucose levels (mg/dL) in the flaps were monitored for a total of 120 minutes. The test animals were euthanized at the 90-minute mark. The results are depicted graphically in FIGS. 1-4.

FIG. 1 is a graph depicting interstitial glucose levels vs. time for all vessel occlusions for all animals tested. Error bars represent standard error of the mean (SEM). As can be seen for the trace for the occluded flaps, interstitial glucose levels dropped steadily and precipitously from the outset, and reached a terminal floor at about the 30-minute time point. In contrast, the glucose levels for both the elevated control flap and the dorsal control remained steady throughout the 90-minute time-course of the experiment while the test animal was alive. Upon euthanasia of the test animals, the glucose levels for the elevated control flap and the dorsal control plummeted. Of note in these data is that the glucose level in the occluded flaps is significantly reduced from controls very early in the experiment, certainly within the first 15 minutes and incontrovertibly within 30 minutes. These data show that the method can be used to detect ischemia very quickly after its onset in an elevated free flap.

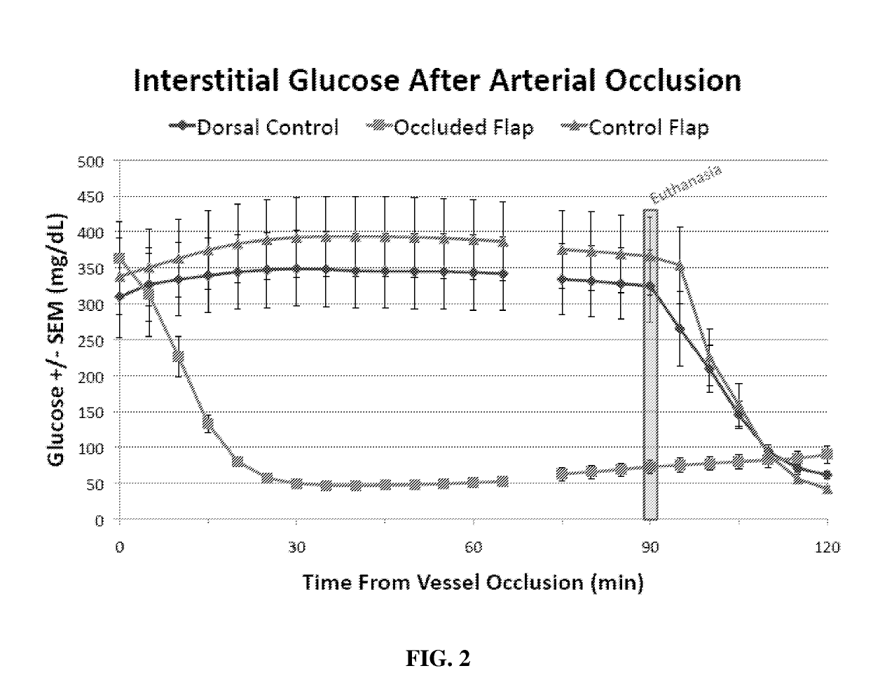

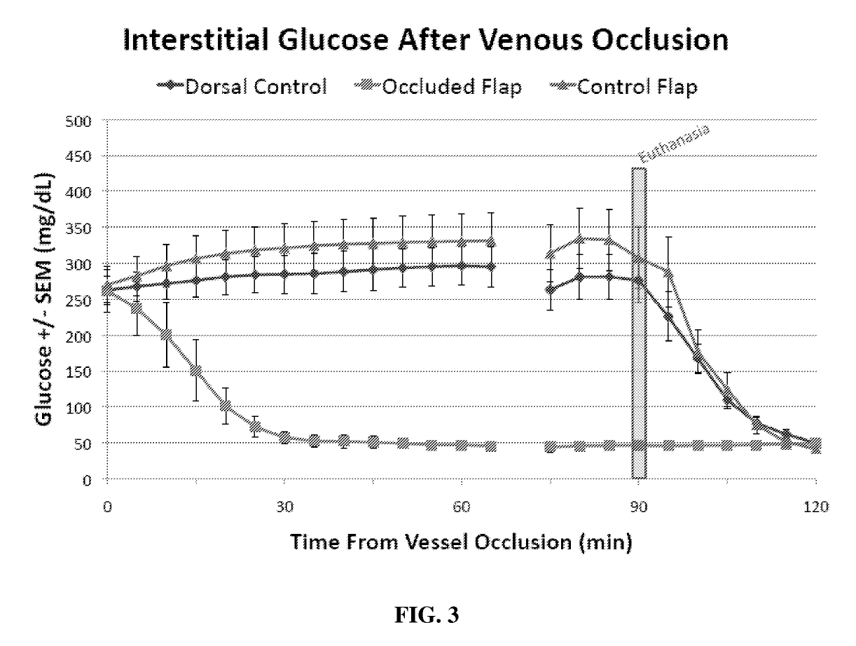

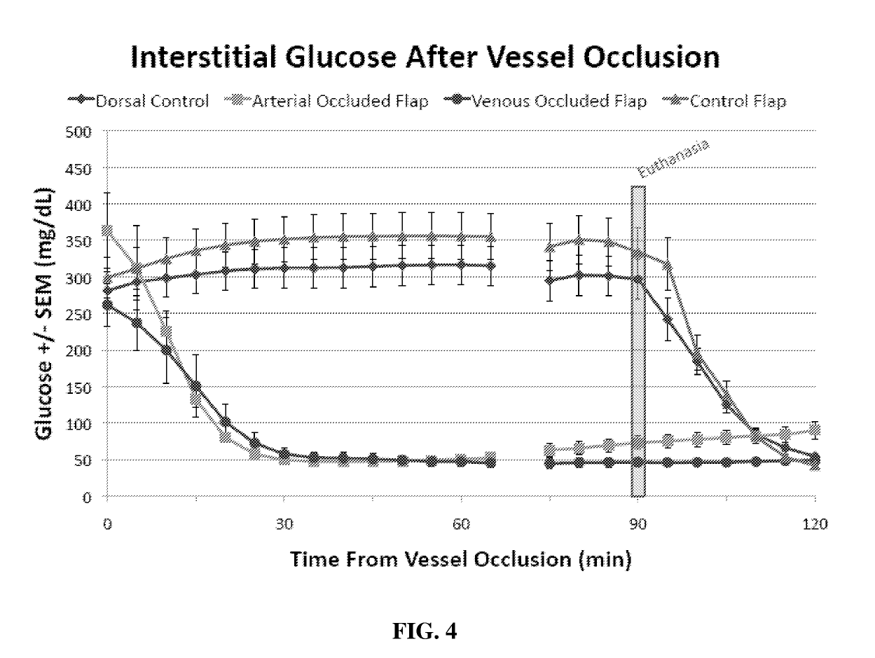

FIGS. 3 and 4 break out the data and present interstitial glucose levels vs. time after arterial occlusion (FIG. 2) and venous occlusion (FIG. 3). Again, glucose levels were monitored in the occluded and control flaps and in a dorsal control for 120 minutes. After 90 minutes, the animals were euthanized. The results here are notable due to the similarity in the plummeting glucose level in the occluded flaps, regardless of whether it was an arterial occlusion or a venous occlusion. These results are very significant in that in the post-surgical setting, early detection of venous occlusions is more difficult because the clinical signs are not as pronounced as in the case of arterial occlusions. Thus, these data show that the method is useful to detect both arterial occlusions and venous occlusions.

FIG. 4 is a graph depicting the two sets of data (arterial occlusion and venous occlusion) overlaid. This graph shows quite convincingly that the present method is useful to detect both arterial and venous occlusions. By at least the 15-minute time point, there is a clear distinction between the test flaps and the controls.

More convincingly, however, are the results when both the glucose levels and their rate of change are analyzed. Here, the method detects ischemia with perfect or near-perfect sensitivity and specificity. The statistics were compiled as shown in Table 1:

TABLE 1
Statistical Analysis

| | | Test | |
|---|---|---|---|
| | | occluded | viable |
| Actual | occluded | a | b |
| | viable | c | d |
| $\begin{matrix}Sensi \text{-} \\ tivity\end{matrix} = \frac{a}{a + b}$ | $\begin{matrix}Speci \text{-} \\ ficity\end{matrix} = \frac{d}{c + d}$ | $PPV = \frac{a}{a + c}$ | $NPV = \frac{d}{b + d}$ |

PPV = Positive predictive value (i.e., given a positive indication of an occlusion, the probability of actually having an occlusion).
NPV = Negative predictive value (i.e., given a negative indication of an occlusion, the probability of not having an occlusion).

To establish a dividing line between what would be considered a glucose level indicating the presence of an occlusion, the blood glucose level in all of the animals was tested via a conventional tail stick. The lowest tail-stick glucose level in the test animals was 118 mg/dL. That value was then used as the cut-off value between occlusion or non-occlusion. That is, a glucose values above 118 g/dL were deemed to indicate the negative condition (no occlusions), while a glucose value below 118 g/dL was deemed to indicate the positive condition (an arterial or venous occlusion). The sensitivity and specificity results using this criterion are shown in Table 2:

TABLE 2
Occlusion Criterion: Glucose <118 mg/dL

| | | Sensitivity | | | |
|---|---|---|---|---|---|
| | | | | Venous | |
| | | All | Arterial | Occlu- | |
| | | Occlusions | Occlusions | sions | Specificity |
| Time | 5 min | | | | 20/21-95.2% |
| after | 10 min | | | | |
| occlu- | 15 min | 5/18-27.8% | 3/10-30.0% | 2/8-25.0% | |
| sion | 20 min | 11/18-61.1% | 7/10-70.0% | 4/8-50.0% | |
| | 25 min | 16/18-88.9% | 10/10-100% | 6/8-75.0% | |
| | 30 min | 18/18-100% | 10/10-100% | 8/8-100% | |

As can be seen from Table 2, the test method was 100% sensitive at 25 minutes for arterial occlusions and 100% sensitive at 30 minutes for venous occlusions. For the entire set of test animals, for all time points used, specificity was 95.2%. Note that these results were achieved using only an absolute number for glucose level. These results did not compare the rate of change of the glucose level, but simply the amount of glucose detected at the indicated time points. These data show that the present method can be used to detect ischemia in transplanted tissue using only a single measurement of glucose taken about 30 minutes after onset of the occlusion.

The next statistical analysis used as a criterion the rate of change in glucose concentration. Here, the negative condition (i.e., no occlusion) was deemed to be a rate of change ≦−2 mg/dL/min. The results are depicted in Table 3:

TABLE 3
Occlusion Criterion:
Rate of Change of Glucose Concentration ≦−2 mg/dL/min.

| | | Sensitivity | | | |
|---|---|---|---|---|---|
| | | | | Occlu- | |
| | | All | Arterial | Venous | |
| | | Occlusions | Occlusions | sions | Specificity |
| Time | 5 min | 6/18-33.3% | 3/10-30.0% | 3/8-37.5% | 16/21-76.2% |
| after | 10 min | 13/18-72.2% | 6/10-60.0% | 7/8-87.5% | |
| occlu- | 15 min | 18/18-100% | 10/10-100% | 8/8-100% | |
| sion | 20 min | 18/18-100% | 10/10-100% | 8/8-100% | |
| | 25 min | 18/18-100% | 10/10-100% | 8/8-100% | |
| | 30 mn | 18/18-100% | 10/10-100% | 8/8-100% | |

As can be seen from Table 3, using rate of change in glucose level is even more sensitive that a simple glucose level taken in time. At the 15-minute time point, the present method was 100% sensitive, for both arterial and venous occlusions. Overall specificity for all data and all time points was 76.2%

The next statistical analysis used as a criterion the rate of change in glucose concentration. But here, the negative condition (i.e., no occlusion) was deemed to include an even steeper rate of change ≦−3 mg/dL/min. The results are depicted in Table 4:

TABLE 4
Occlusion Criterion:
Rate of Change of Glucose Concentration ≦−3 mg/dL/min.

| | | Sensitivity | | | |
|---|---|---|---|---|---|
| | | | | Venous | |
| | | All | Arterial | Occlu- | |
| | | Occlusions | Occlusions | sions | Specificity |
| Time | 5 min | 6/18-33.3% | 3/10-30.0% | 3/8-37.5% | 18/21-85.7% |
| after | 10 min | 12/18-66.7% | 6/10-60.0% | 6/8-75.0% | |
| occlu- | 15 min | 18/18-100% | 10/10-100% | 8/8-100% | |
| sion | 20 min | 18/18-100% | 10/10-100% | 8/8-100% | |
| | 25 min | 18/18-100% | 10/10-100% | 8/8-100% | |
| | 30 mn | 18/18-100% | 10/10-100% | 8/8-100% | |

Again, as can be seen from Table 4, with the criterion of ≦−3 mg/dL/min rate of change indicating no occlusion, the present method was 100% sensitive, for both arterial and venous occlusions at the 15-minute time point. Overall specificity for all data and all time points was 85.7%.

Comparable results were also found when the statistical analysis used as a criterion a rate of change in glucose concentration of ≦−4 mg/dL/min. The results are depicted in Table 5:

TABLE 5
Occlusion Criterion:
Rate of Change of Glucose Concentration ≦−4 mg/dL/min.

| | | Sensitivity | | | |
|---|---|---|---|---|---|
| | | | | Venous | |
| | | All | Arterial | Occlu- | |
| | | Occlusions | Occlusions | sions | Specificity |
| Time | 5 min | 3/18-16.7% | 1/10-10.0% | 2/8-25.0% | 20/21-95.2% |
| after | 10 min | 11/18-61.1% | 6/10-60.0% | 5/8-62.5% | |
| occlu- | 15 min | 17/18-94.4% | 10/10-100% | 7/8-87.5% | |
| sion | 20 min | 18/18-100% | 10/10-100% | 8/8-100% | |
| | 25 min | 18/18-100% | 10/10-100% | 8/8-100% | |
| | 30 mn | 18/18-100% | 10/10-100% | 8/8-100% | |

Here, with the criterion of ≦−4 mg/dL/min rate of change indicating no occlusion, the present method was 100% sensitive for arterial occlusions at the 15-minute time point. 100% sensitivity for venous occlusions was achieved at the 20-minute time point. Overall specificity for all data and all time points was 95.2%.

Combining data for all occlusions and using two criteria (<118 mg/dL absolute glucose concentration to indicate occlusion, plus rate of change of glucose concentration ≦−2 mg/dL/min to indicate no occlusion) yielded the following results:

TABLE 6
Occlusion Criterion:
Glucose <118 mg/dL and Rate of Change of
Glucose Concentration ≦−2 mg/dL/min.

| | | Sensitivity | | | |
|---|---|---|---|---|---|
| | | | Arterial | | |
| | | All | Occlu- | Venous | |
| | | Occlusions | sions | Occlusions | Specificity |
| Time after | 5 min | 0/18-0% | | | 21/21-100% |
| occlusion | 10 min | 2/18-11.1% | | | |
| | 15 min | 5/18-27.8% | | | |
| | 20 min | 11/18-61.1% | | | |
| | 25 min | 16/18-88.9% | | | |
| | 30 min | 18/18-100% | | | |

Here, the statistical results show 100% sensitivity at the 30-minute time point, with 100% specificity (no false positives, no false negatives). This analysis quite clearly shows the utility of the present invention for detecting ischemia in transplant tissue.

Lastly, an analysis was done using alternative criteria: <118 mg/dL absolute glucose concentration to indicate occlusion, or rate of change of glucose concentration ≦−4 mg/dL/min to indicate no occlusion. The results are shown in Table 7.

TABLE 7
Occlusion Criterion: Glucose <118 mg/dL or Rate of Change of Glucose
Concentration ≦−4 mg/dL/min.

| | | Sensitivity | | | |
|---|---|---|---|---|---|
| | | | | Venous | |
| | | All | Arterial | Occlu- | |
| | | Occlusions | Occlusions | sions | Specificity |
| Time | 5 min | 3/18-16.7% | 1/10-10.0% | 2/8-25.0% | 19/21-90.5% |
| after | 10 min | 11/18-61.1% | 6/10-60.0% | 5/8-62.5% | |
| occlu- | 15 min | 17/18-94.4% | 10/10-100% | 7/8-87.5% | |
| sion | 20 min | 18/18-100% | 10/10-100% | 8/8-100% | |
| | 25 min | 18/18-100% | 10/10-100% | 8/8-100% | |
| | 30 min | 18/18-100% | 10/10-100% | | |

Using these alternative criteria, the present method was 100% sensitive at the 15-minute time point for arterial occlusions and 100% sensitive at the 20-minute time point for venous occlusions. Specificity overall was 90.5%.

Cumulatively, these data show that the present method can detect and monitor ischemia (due to venous and/or arterial occlusions) using absolute glucose concentration and/or rate of change in glucose concentration as a metric.

What is claimed is:

1. A method of detecting and/or monitoring ischemia in a tissue or organ, the method comprising:
measuring in real time or near-real time interstitial glucose concentration, or rate of change of interstitial glucose concentration over time, or both, in the tissue or organ, wherein a reduced glucose concentration or a negative rate of change of glucose concentration in the tissue or organ as compared to a control glucose concentration or rate of change indicates ischemia in the tissue or organ.

2. The method of claim 1, comprising measuring interstitial glucose concentration.

3. The method of claim 2, wherein a measured interstitial glucose concentration of less than about 118 mg/dL indicates ischemia in the tissue or organ.

4. The method of claim 1, comprising measuring rate of change of interstitial glucose concentration over time.

5. The method of claim 4, wherein a measured rate of change of interstitial glucose concentration steeper than about −2 mg/dL/minute indicates ischemia in the tissue or organ.

6. The method of claim 4, wherein a measured rate of change of interstitial glucose concentration steeper than about −3 mg/dL/minute indicates ischemia in the tissue or organ.

7. The method of claim 4, wherein a measured rate of change of interstitial glucose concentration steeper than about −4 mg/dL/minute indicates ischemia in the tissue or organ.

8. The method of claim 1, comprising measuring interstitial glucose concentration and rate of change of interstitial glucose concentration over time.

9. The method of claim 8, wherein a measured interstitial glucose concentration of less than about 118 mg/dL and a measured rate of change of interstitial glucose concentration steeper than about −2 mg/dL/minute indicates ischemia in the tissue or organ.

10. The method of claim 9, wherein a measured rate of change of interstitial glucose concentration steeper than about −3 mg/dL/minute indicates ischemia in the tissue or organ.

11. The method of claim 9, wherein a measured rate of change of interstitial glucose concentration steeper than about −4 mg/dL/minute indicates ischemia in the tissue or organ.

12. The method of claim 1, wherein the tissue or organ is an autologous free tissue transfer.

13. A method of detecting and/or monitoring ischemia in a free tissue transfer, the method comprising:
measuring in real time or near-real time interstitial glucose concentration, or rate of change of interstitial glucose concentration over time, or both, in the free tissue transfer, wherein a reduced glucose concentration or a negative rate of change of glucose concentration in the free tissue transfer as compared to a control glucose concentration or rate of change indicates ischemia in the free tissue transfer.

14. The method of claim 13, comprising measuring interstitial glucose concentration.

15. The method of claim 14, wherein a measured interstitial glucose concentration of less than about 118 mg/dL indicates ischemia in the free tissue transfer.

16. The method of claim 13, comprising measuring rate of change of interstitial glucose concentration over time.

17. The method of claim 16, wherein a measured rate of change of interstitial glucose concentration steeper than about −2 mg/dL/minute indicates ischemia in the free tissue transfer.

18. The method of claim 17, wherein a measured rate of change of interstitial glucose concentration steeper than about −3 mg/dL/minute indicates ischemia in the free tissue transfer.

19. The method of claim 17, wherein a measured rate of change of interstitial glucose concentration steeper than about −4 mg/dL/minute indicates ischemia in the free tissue transfer.

20. The method of claim 13, comprising measuring interstitial glucose concentration and rate of change of interstitial glucose concentration over time.

21. The method of claim 20, wherein a measured interstitial glucose concentration of less than about 118 mg/dL and a measured rate of change of interstitial glucose concentration steeper than about −2 mg/dL/minute indicates ischemia in the free tissue transfer.

22. The method of claim 21, wherein a measured rate of change of interstitial glucose concentration steeper than about −3 mg/dL/minute indicates ischemia in the free tissue transfer.

23. The method of claim 21, wherein a measured rate of change of interstitial glucose concentration steeper than about −4 mg/dL/minute indicates ischemia in the free tissue transfer.
